(19) United States
(12) Patent Application Publication
(10) Pub. No.: US 2022/0406421 A1
(43) Pub. Date: Dec. 22, 2022

(54) SYSTEMS AND METHODS FOR EARLY DETECTION AND MANAGEMENT OF CLINICAL CRITICAL EVENTS

(71) Applicant: THE CHILDREN'S HOSPITAL OF PHILADELPHIA, Philadelphia, PA (US)

(72) Inventors: Victor M. RUIZ, Philadelphia, PA (US); Lingyun SHI, Philadelphia, PA (US); Fan MI, Philadelphia, PA (US); Fuchiang TSUI, Philadelphia, PA (US)

(73) Assignee: THE CHILDREN'S HOSPITAL OF PHILADELPHIA, Philadelphia, PA (US)

(21) Appl. No.: 17/776,955

(22) PCT Filed: Nov. 13, 2020
(86) PCT No.: PCT/US2020/060447

§ 371 Date: May 13, 2022

Related U.S. Application Data
(60) Provisional application No. 62/936,191, filed on Nov. 15, 2019.

(51) Int. Cl. G16H 10/60; G16H 50/20; G16H 40/63; A61B 5/00; G06F 40/40

(52) U.S. Cl. CPC G16H 10/60; G16H 50/20; G16H 40/63; A61B 5/7264; G06F 40/40

(57) ABSTRACT

The present disclosure relates to systems and methods for early detection and management of clinical critical events.

### CROSS-REFERENCE TO RELATED APPLICATIONS

This application claims priority to U.S. Provisional Patent Application Ser. No. 62/936,191, filed Nov. 15, 2019, the entire contents of which are incorporated herein by reference.

### BACKGROUND INFORMATION

As discussed in further detail below, exemplary embodiments of the present disclosure address shortcomings of existing systems and provide notable benefits in comparison to such systems.

Common challenges in ICUs include high mortality rates compared with other units (10%-29% in adult ICUs; 2%-6% in pediatric ICUs), increased admissions, medical errors, intensivist shortage, and alert fatigue [32]. In the wake of the COVID-19 pandemic, telehealth can be of value to hospitals with limited intensivist resources, e.g., monitoring patient streaming waveforms remotely.

Current state-of-the-art research demonstrated advantages in using machine learning and streaming data in intensive care [1]. However, extant research is limited on implementing machine learning models in clinical practice.

Early detection of a patient's critical events enables clinicians' early intervention to prevent subsequent morbidity and mortality [2,3]. Current early warning systems (EWS) still face several general issues. First, there is limited development of pediatric EWS for critical events and the performance of direct deployment of adult EWS to pediatric population is lower than in the adult population [4-6]. Second, there is very limited critical event prediction in pediatric intensive care for high-risk congenital heart disease groups such as single-ventricle patients. To the inventors' knowledge, the model by Rusin et. al. [7] is the only model to predict critical events in real time among single ventricle (SV) patients (25 SV patients with 20 critical events) with an accuracy 91% at 1-hour prior to an event but a much lower performance of 50-60% at 3 or more hours prior to an event. Moreover, it only uses partial high-speed-data (HSD)—ECG waveform—for analysis without any commonly available electronic health record (EHR) data. Other SV models can only provide one-time static event prediction following surgery (using only surgical related factors) without considering factors observed in intensive care. Third, most EWS development including commercial solutions only use HSD or EHR data in isolation, which limits its accuracy. Fourth, current predictive modeling using complicated models such as deep neural networks or random forest are often regarded as “black-box” models that are difficult to explain or interpret. This raises the issue of trust for end users like clinicians. Predictive models alone are unlikely to have clinical impact unless clinicians can understand and trust the models to make decisions [8]. Thus, current EWS including commercial solutions still face those aforementioned issues. Other issues such as shortage of intensivists, alert fatigue, and high costs of patient care further stress the need for pediatric EWS.

There are several fundamental needs and issues with high-speed waveform data collection, storage and processing at typical health care facilities, such as The Children's Hospital of Philadelphia (CHOP). For example, there is often a lack of real-time pediatric EWS in intensive care for the high-risk SV population. Second, there is often a lack of HSD research environment that can provide comprehensive collection, permanent data storage, and flexible queries of HSD data. Some Commercial solutions are available to collect waveform data, including Nuvon by Bernoulli® and BedmasterEx™ by Excel Medical. Nuvon automatically collects HSD from most of beds without a manual configuration but it typically only retains data up to the last 100 days while purging older data. BedmasterEx™ collects HSD from a small portion of beds (up to 150 beds) at CHOP. Both systems provide a graphical user interface (GUI) to pull data. Their GUIs only allow users to pull a small portion of data at a time (few seconds to minutes). A substantial labor-intensive effort is required if a longer period of historical data (hours to days) is needed.

Another ad-hoc system is Moberg (by Moberg Research, Inc), which relies on a USB drive to collect waveform data. This modality, however, raises concerns for patient data security and for storage scalability. Third, there is a lack of research-to-practice translational environment for HSD that can translate advanced HSD research to clinical practice. The translational environment should include (1) an integrated data warehouse comprising various data types including EHRs and HSD and (2) a deployment platform that collects (predictive) models from researchers or end users and enables the deployment of the models to real-time streaming data. HSD research is getting more traction given current hardware and software advancement and an urgent need for ICUs to adopt new research-supported technology to enhance current practice. However, commercial solutions often have a closed environment to hinder such deployment and algorithm integration. For example, it would be challenging to apply a researcher's signal processing algorithm (e.g., delineation of electrocardiogram [ECG] waveforms) to real-time waveform data with currently available solutions.

It is further noted that congenital heart defects (CHD) affect nearly 40,000 births in the US annually. Twenty five percent of CHD births are critical (CCHD), and require invasive procedures in the first year of life. CCHD outcomes are significantly worse than those of CHD babies. Single-ventricle (SV) physiology is among the most severe CCHDs, and has increased morbidity and mortality in the peri-operative period before stage-2 palliation. Critical events such as extracorporeal membrane oxygenation (ECMO), intubation, and cardiac arrest are important precursors of patient deterioration.

### SUMMARY

Exemplary embodiments of the present disclosure comprise systems and methods for addressing several issues, including those stated in the background information section. First, exemplary embodiments address the problem of real-time collecting, storing, and processing HSD at health care facilities such as CHOP. The capability of processing HSD prospectively enables clinicians to detect and intervene potential critical events; retrospective data analysis enables the development of advanced machine learning methods by researchers. Recent research sheds light on the importance of high-speed data acquisition and analysis. In 2018, Dr. Simpao et. al. [9] at CHOP reported that a laryngospasm event in a 21-month old child was able to be observed through pulse oximetry (SaO2) data recorded in middleware medical device integration (MDI) by Nuvon whereas the same SaO2 data collected in Epic through the Anesthesia Information Management System (AIMS) did not show such event. During the laryngospasm event, the SaO2 collected by MDI showed the drop of oxygen below 60% but the SaO2 in Epic only showed a minor dip with oxygen level still above 80%. The discrepancy was due to different sampling frequencies, i.e., MDI has a sampling frequency at the second level whereas Epic system only stores data samples at the minute level. Thus, the quality of data collection can potentially limit the capability of identifying a clinical event, which can be in the future automatically identified or prevented in real-time by machine learning or AI methods the inventors develop. Moreover, Rajpurkar et. al. [10] at Stanford University in 2017 developed an algorithm that exceeds the performance of board-certified cardiologists in detecting wide range of heart arrhythmias from ECG waveforms. From the Simpao et. al. study, it becomes critical to develop a real-time monitor system that has access to the high-speed data in order to detect and eventually predict such critical events.

Second, it addresses the lack of data integration between HSD and EHR data for predictive modeling. Integrating both HSD and EHR enables the predictive system to have comparable information received by clinicians in order to reduce false positives and increase sensitivity. Third, it addresses the problem of limited development in predictive modeling for high-risk patients with single-ventricle physiology. Currently only very limited research using HSD (e.g., Rusin et. al). Fourth, it addresses the dilemma of lack of explanation of predictive models. Having the capability to explain how risk is estimated would enhance the trust from clinicians and other end users. Fifth, it addresses the problem of lack of system integration between EHR system (e.g., Epic) and HSD/modeling system. Such system integration would facilitate the deployment of decision support solutions that fit into clinicians' workflow without the need for users to spend time logging onto a separate system or reentering patient information. With the advent of EHRs, artificial intelligence, and fast speed of computer processors and disk storage, there is an increasing trend to develop a real-time decision support system that uses both HSD and EHR data [11,12].

Exemplary embodiments of the present disclosure, sometimes referred to herein as the “I-WIN” system, include a real-time predictive system for critical events in intensive care, which is unique, innovative and comprehensive. I-WIN, integrating innovative medical informatics research technologies and industry standards, has the following innovative functions:

1. Performing bedside-monitor high-speed data acquisition and signal processing analytics through a distributed platform;

2. Integrating both high-speed data and electronic health records for risk prediction;

3. Retrieving structured and unstructured (free-text) electronic health records from Epic EHR system;

4. Performing natural language processing for extraction of clinical concepts from narrative clinical reports;

5. Supporting precision predictive modeling for specific high-risk populations (e.g., patients with single-ventricle physiology);

6. Providing real-time patient risk index to clinicians;

7. Explaining predicted risk (e.g., high vs. low) based on underlying patient-specific risk factors to enable trust from end users; and

8. Integrating graphical user interface into Epic EHR system to better fit clinicians' workflow.

I-WIN can assist end users (e.g., clinicians and house staff) to achieve early and accurate prediction of clinical critical events for patients in intensive care by leveraging high-speed data, EHR data (from Epic), artificial intelligence, and expert clinical knowledge

Exemplary embodiments of the present disclosure will facilitate early intervention and reduction of clinical critical events, morbidity and mortality, and hospital costs, and improve the quality of patient care and patient safety. The inventors proposed work supports the scalability to potentially collect and process hospital-wide bedside monitors (˜800) and to predict critical events for different hospital populations, and it has potential commercialization opportunity for other hospitals that would like to deploy such system.

With the advent of big data in healthcare and the need for early detection and intervention of patient condition deterioration, the I-WIN system can process both streaming clinical high-speed data (HSD) and electronic health records (EHRs) for early prediction and management of clinical critical events (CEs) in cardiac intensive care. These include extracorporeal membrane oxygenation (ECMO), emergent endotracheal intubation (EEI), and cardiac arrest (CA) with prediction horizons ranging from 8 hours to 1 hour in advance, based on the inventors' recent work [1].

The development of early prediction of critical events using realtime waveform and EHR data are critical in intensive care and potentially applied to peri-operative and inter-operative monitoring. The median cost for hospital stay after the institution of ECMO was $156,324 per pediatric patient.[24] For surviving ventilated patients, median costs for newborns with ≤32 weeks' gestation were $51,000-$209,000, whereas median costs for older patients (33+ weeks of gestational age and children) were $17,000-$25,000.[25] The average costs for cardiac arrest of pediatric patients during their hospital stay were about $30,000 US dollars from an England study.[26]

Exemplary embodiments provide significant potential cost savings. In addition, exemplary embodiments address issues related to alert fatigue. For example, existing bedside monitors use simple threshold or univariate-based alert approach creates many false alarms due to its lack of taking into account a patient's overall conditions. However, while reducing alert fatigue, the inventors will consider other metrics such as sensitivity, and having the capability to process multiple sources of data to increase the sensitivity of detecting events while keeping high specificity (low false alarms).

Exemplary embodiments also provide for data integration. Unlike extant state-of-the-art approaches focusing on only one/few types of data, the I-WIN is capable of linking EHR data with streaming waveform data to increase the prediction accuracy while reducing false alarms. Moreover, I-WIN has the capability to incorporate clinicians' years of clinical domain knowledge in conjunction with the big clinical data.

Exemplary embodiments also provide a potential positive impact to current practice. For example, currently at CHOP, the Division of Cardiothoracic Anesthesiology provides anesthesia and pain management more than 3,000 surgeries each year;[27] the Division of Cardiology serves more than 32,000 outpatient visits with more than 69,000 tests including ECG waveforms.[28] They all can benefit from the inventors' proposed system for waveform analytics and/or event detection.

I-WIN (Intensive Care Warning Index) can perform numerous functions, including providing an index/score/flag for potential clinical events (unplanned intubation, ECMO, cardiac arrest) and mortality. In addition, I-WIN can provide ICU admission and readmission prediction and management. It can also provide risk explanation, transfer learning to self-adjust to a new hospital population or the local hospital over time, and provide intervention strategies based on patient-specific risk factors.

Covered patients can include pediatric/adult in ICU, and patients outside ICUs with a risk to be transferred to ICU.

Exemplary embodiments include a method for early detection and management of clinical critical events, where the method comprises: analyzing high-speed bedside waveform data; analyzing EHR data; and predicting critical events (CE) based on the analysis of the high-speed bedside waveform data and the analysis of the EHR data. In certain embodiments, analyzing high-speed bedside waveform data and EHR data comprises one or more of the following: computer vision, transfer learning, explanation modeling machine learning, natural process learning, distributed processing, generalizability learning or waveform processing. In particular embodiments, the waveform processing comprises real-time waveform data acquisition.

Specific embodiments include a system for early detection and management of clinical critical events, where the system comprises: a module configured for high performance data landing or storage; a module configured for distributed data processing; a module configured for data science monitoring; and a module configured for real-time data visualization. In some embodiments, the module configured for high performance data landing or storage comprises a scale-out storage solution and a NoSQL/SQL database. In certain embodiments, the module configured for distributed data processing is configured to perform waveform analysis, natural language processing or machine learning feature extraction. In particular embodiments, the module configured for data science monitoring comprises a knowledge base and an inference engine. In some embodiments, the module configured for real-time data visualization comprises a secure web-based user interface and a business intelligence (BI) platform interface.

Certain embodiments include a system for early detection and management of clinical critical events, where the system comprise: a data source layer; an extract, transform and load (ETL) layer; a distributed artificial intelligence (AI) layer; and a presentation layer. In particular embodiments, the data source layer is configured to transfer data to the ETL layer; the ETL layer is configured to transfer data to the distributed AI layer; and the distributed AI layer is configured to transfer data to the presentation layer. In some embodiments, the data source layer comprises medical devices and bedside monitors. In particular embodiments, the ETL layer includes streaming data acquisition from the bedside monitors and the medical devices. In specific embodiments, the data source layer comprises: an electronic health record (EHR) system; an administrative information system; and a clinical information system. In certain embodiments, the ETL layer is configured to apply natural language processing to data transferred from the EHR system, the administrative information system and the clinical information system.

In particular embodiments, the ETL layer is configured to clean data from the data source layer via natural language processing. In some embodiments, the ETL layer is configured to merge records from the data source layer via natural language processing. In specific embodiments, the ETL layer is configured to apply business rules to the data source layer via natural language processing. In certain embodiments, the distributed AI layer comprises a hybrid database cluster. In some embodiments, the distributed AI layer comprises predictive models and an inference engine configured to communicate with the hybrid database cluster. In specific embodiments, the distributed AI layer is configured to apply signal processing the streaming data acquisition from the ETL layer. In certain embodiments, the presentation layer comprises a web application and graphical user interface.

In the present disclosure, the term “coupled” is defined as connected, although not necessarily directly, and not necessarily mechanically.

The use of the word “a” or “an” when used in conjunction with the term “comprising” in the claims and/or the specification may mean “one,” but it is also consistent with the meaning of “one or more” or “at least one.” The term “about” means, in general, the stated value plus or minus 5%. The use of the term “or” in the claims is used to mean “and/or” unless explicitly indicated to refer to alternatives only or the alternative are mutually exclusive, although the disclosure supports a definition that refers to only alternatives and “and/or.”

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements, possesses those one or more steps or elements, but is not limited to possessing only those one or more elements. Likewise, a step of a method or an element of a device that “comprises,” “has,” “includes” or “contains” one or more features, possesses those one or more features, but is not limited to possessing only those one or more features. Furthermore, a device or structure that is configured in a certain way is configured in at least that way, but may also be configured in ways that are not listed.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will be apparent to those skilled in the art from this detailed description.

### BRIEF DESCRIPTION OF THE FIGURES

FIG. 1 illustrates a schematic of the architecture of an exemplary embodiment of the Intensive-care Warning Index (I-WIN) system according to the present disclosure.

FIG. 2 illustrates a schematic of a WIN informatics framework.

FIG. 3 is example of an I-WIN tele-health real-time single patient waveform view.

FIG. 4 is an exemplary real-time multi-patient waveform view for multiple patients.

FIG. 5 illustrates an exemplary view of single patient bundled view and risk estimate.

FIG. 6 illustrates a summary of the features provided in the I-WIN system in contrast to other systems.

FIG. 7 illustrates a predictive model performance for I-WIN and other systems.

FIG. 8 illustrates an example of a high-speed data acquisition system from bedside monitors for the I-WIN system.

FIG. 9 illustrates I-WIN spO2 data quality closely matches data collected manually with clinical charts.

FIG. 10 illustrates I-WIN heart rate data quality closely matches data collected manually with clinical charts

FIG. 11 illustrates a schematic of an I-WIN customizable streaming (artificial intelligence) AI platform.

FIG. 12 illustrates a schematic of an I-WIN network diagram.

FIG. 13 illustrates a schematic of a hierarchical high speed (HS) data structure

FIG. 14 illustrates exemplary embodiments provide data protection in the forms of raw data backup, replication failover protection and database storage backup.

FIG. 15 illustrates an example of coding for I-WIN hierarchical data structure.

FIG. 16 illustrates spO2 data for other systems does not match as closely with manual collected data in comparison to exemplary embodiments of the present disclosure.

FIG. 17 illustrates I-WIN prediction performance at five different prediction time windows (from 1 to 8 hours prior to a critical event.

FIG. 18 illustrates an overview of an exemplary embodiment of the Intensive-care Warning Index (I-WIN) system according to the present disclosure.

FIG. 19 illustrates an exemplary embodiment of the I-WIN architecture comprising real-time high-performance data collection, storage and data integration, data processing, data analytics, and data visualization.

FIG. 20 illustrates a graph of an ECG signal processing algorithm based on hidden Markov model for the detection of six ECG waves: P, Q, R, S, T, and U, according to exemplary embodiments of the present disclosure

FIG. 21 illustrates a table of the accuracy of ECG algorithm using Hidden Markov Model based on the data from QT ECG annotated database within the range of 40 ms from each annotated peak.

FIG. 22 illustrates the survival probability for persons with CCHD versus noncritical CHDs over different ages.

FIG. 23 illustrates the proportion free from death and transplant versus time as noted.

FIG. 24 illustrates data retrieved from the Children's Hospital of Philadelphia (CHOP) during a retrieval period, inclusion and cases and controls.

FIG. 25 illustrates a flowchart of one embodiment including parameter optimization, feature extraction, feature selection, and model training.

FIGS. 26-28 are published from Shahar [30] and illustrate Frequent Temporal Patterns (FTPs).

FIG. 26 illustrates temporal abstractions, e.g. with gradient (change), slope, time since measurement, low/normal/high.

FIG. 27 shows multivariate state sequences (MSS) as noted.

In FIG. 28, the Frequent Temporal Pattern (FTP) is shown for heart rate and oxygen saturation. The state intervals' start/end times are abstracted into temporal relationships between states (before, co-occur, after).

FIG. 29 is published form Valka [31] and illustrate trend (temporal features) as noted.

FIGS. 30-33 display results of exemplary embodiments, including calibration curves of FTP models at different times before critical events.

FIG. 34 displays characteristics of a best FTP model, and FIG. 35 displays last values, trends and FTP as noted.

### DETAILED DESCRIPTION OF ILLUSTRATIVE EMBODIMENTS

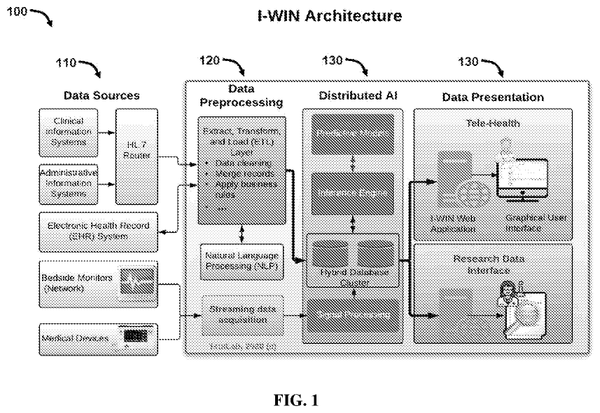

Referring initially to FIG. 1 a schematic of the architecture of an exemplary embodiment of a system 100 (also referred to herein as an “Intensive-care Warning Index” [I-WIN] system) according to the present disclosure is provided. As shown in FIG. 1, system 100 architecture includes data source layer 110, an extract, transform and load (ETL) layer 120, a distributed artificial intelligence (AI) layer 130, and a presentation layer 140. Components and functional aspects of each layer are illustrated in FIG. 1.

In the embodiment shown, data source layer 110 feeds real-time data to I-WIN, which comprises conventional electronic health record system (Epic), and high-speed medical devices—with data sampling rates ranging from 0.5 Hz to 240 Hz—such as bedside monitors and ventilators. ETL layer 120 manages streaming data acquisition, real-time data transformation, and scalable data loading for both EHR and high-speed medical device data.

Distributed AI layer 130 comprises natural language processing (NLP) component for processing unstructured electronic health record (HER) data, an inference engine for running the models, a signal processing component for extracting waveform features (e.g., ST segments), and machine-learning predictive models for predicting deterioration. In the embodiment shown, hybrid databases are adopted for storing waveform and EHR data. Distributed open-source platforms are used for data processing (Apache Spark) and caching to achieve scalability. In the embodiment shown, presentation layer 140 comprises a secure web-based interface that allows intensivists to remotely monitor patients.

The accuracy of system 100 was evaluated in data collection and deterioration prediction. I-WIN had 100% waveform data accuracy measured by mean square errors by comparing with an FDA-approved waveform monitor system. A random-forest predictive model was also evaluated using a cohort of 766 single-ventricle patients at CHOP and the average 5-fold the area under the ROC curve (AUC) was 0.93 (95% CI: 0.9-0.96) with four hours before an event. I-WIN is being deployed at CHOP with pilot clinician users to further improve its usability and system interface.

Clinical care in today's setting provides unique challenges, including the burden of massive information (e.g. COVID-19), alert fatigue, inexperience and under staffing, and increased visits and costs. In addition, there is a lack of scalable architecture for data collection from medical devices, lack of waveform data query platform and research and lack of real-time clinical decision support development platform.

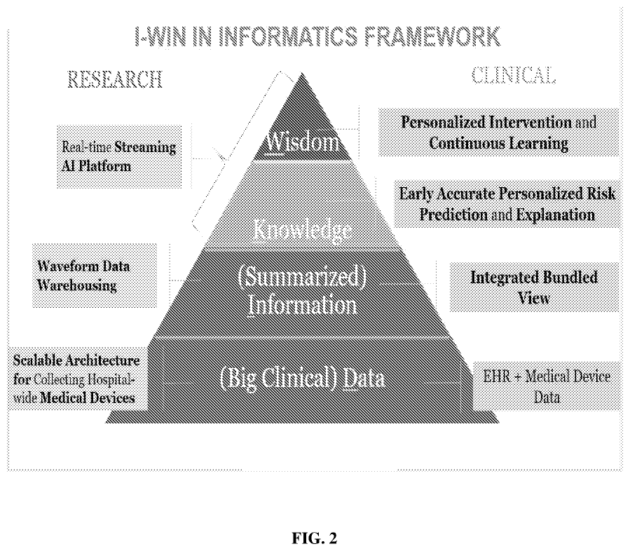

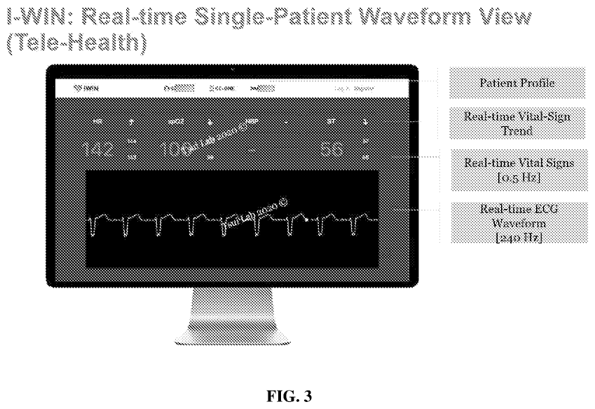

The I-WIN informatics framework illustrated in FIG. 2 provides a solution to these issues. Research information in the framework includes real-time streaming AI platform, waveform data warehousing and scalable architecture for collecting hospital-wide medical devices. Clinical information includes personalized intervention and continuous learning, early accurate personalized risk prediction and explanation, integrated bundled view and combined EHR and medical-device data. Clinical users of I-WIN include intensivists, clinicians and nurses while research users include data scientists, data engineers and researchers.

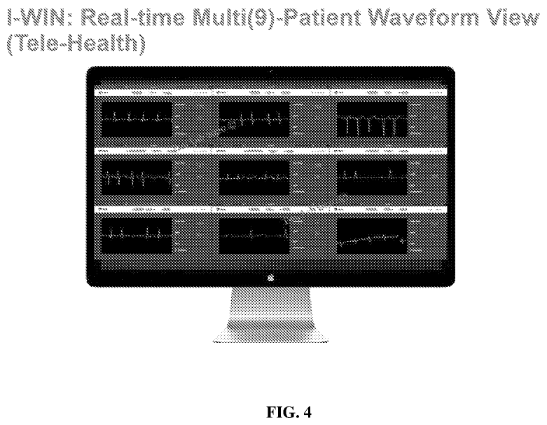

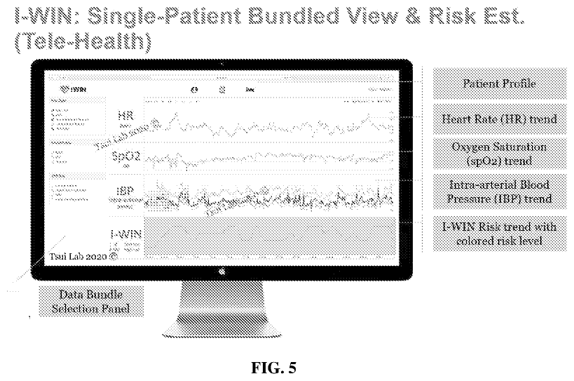

An example of an I-WIN tele-health real-time single patient waveform view is shown in FIG. 3. This view provides the patient profile, real-time vital sign trend, real time vital signs and real-time ECG waveform. It is understood that other exemplary views may provide a different combination of parameters. FIG. 4 provides an exemplary real-time multi-patient waveform view for nine separate patients.

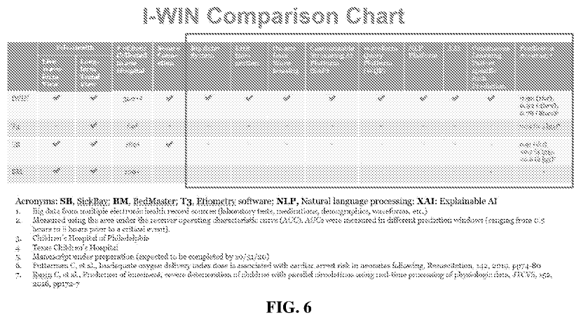

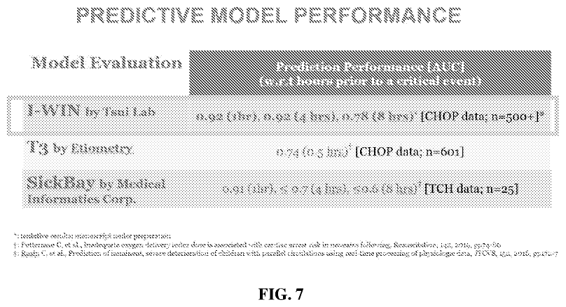

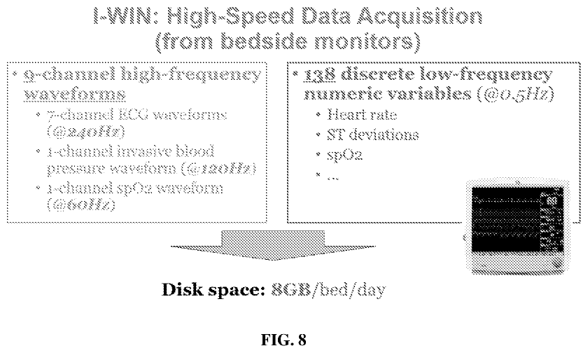

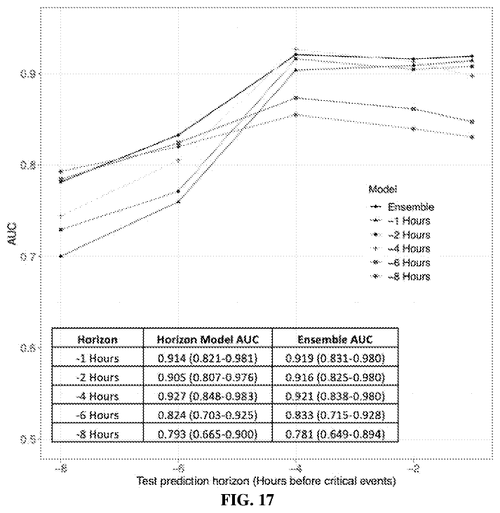

I-WIN can provide risk prediction of critical events, including for example, cardiac arrest, intubation and extra corporeal membrane oxygenation that can be categorized in different risk levels (e.g. low, moderate or high). FIG. 5 provides an exemplary view of single patient bundled view and risk estimate. A comparison chart shown in FIG. 6 provides a summary of the features provided in the I-WIN system in contrast to other systems, and predictive model performance for I-WIN and other systems is shown in FIG. 7. I-WIN prediction performance at five different prediction time windows (from 1 to 8 hours prior to a critical event) is shown in FIG. 17.

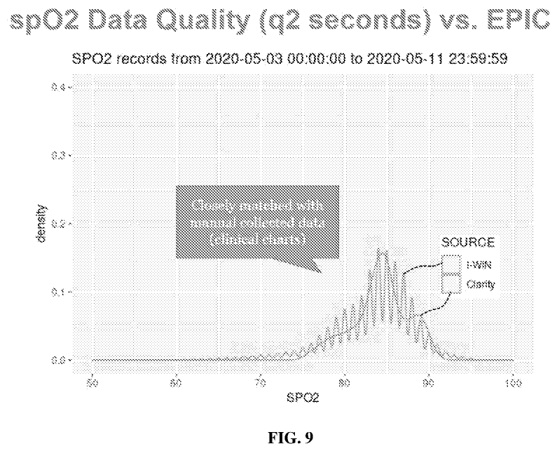

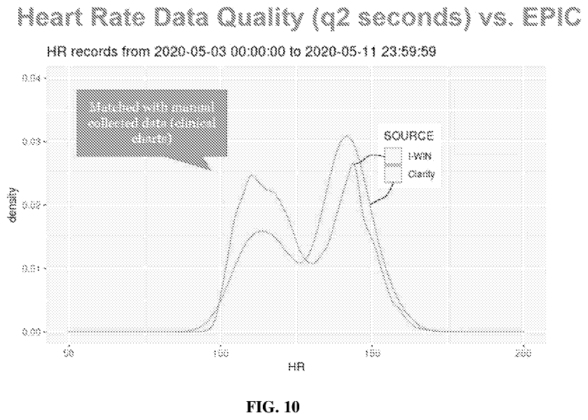

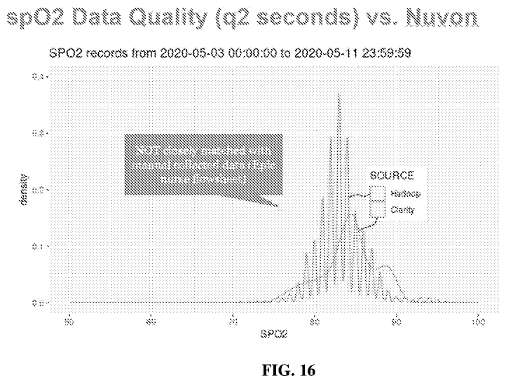

FIG. 8 provides an example of a high-speed data acquisition system from bedside monitors for the I-WIN system. In one analysis, I-WIN waveform data quality (240 Hz) was compared to BedMaster® data, with one day's worth of waveform data from I-WIN and FDA-approved BedMaster® that both collected data from GE bedside monitors. The mean-square-error (MSE) was calculated to be zero between the two signals, (i.e., no errors were found in I-WIN). As shown in FIG. 9 and FIG. 10, I-WIN spO2 and heart rate data quality (q2 seconds) was shown to closely match data collected manually with clinical charts. FIG. 16 shows a comparison of spO2 data for other systems does not match as closely with manual collected data.

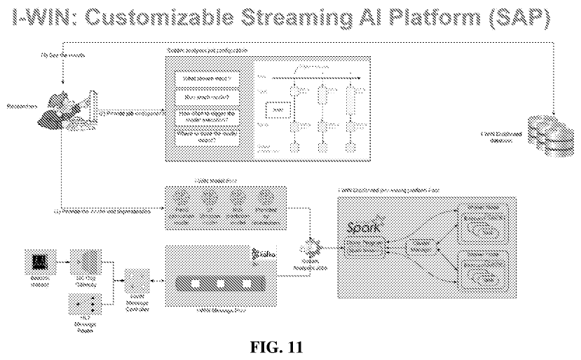

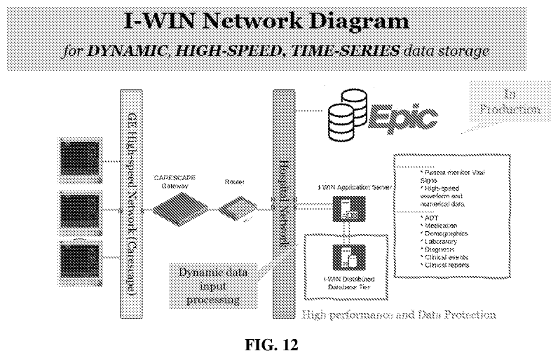

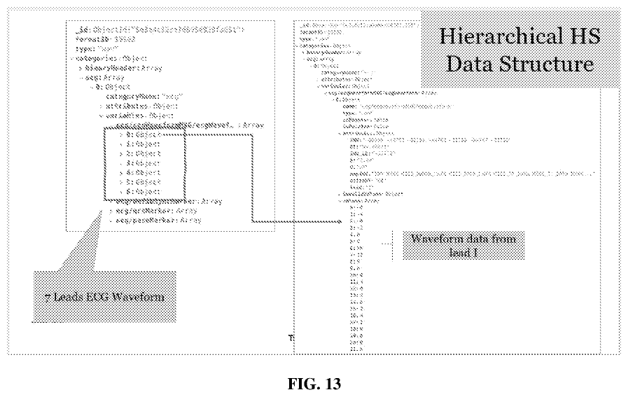

FIG. 11 provides a schematic of an I-WIN customizable streaming (artificial intelligence) AI platform that provides a model and dependencies from a model pool, allows a user to provide a job configuration and allows the user to see the results from a distributed database. An I-WIN network diagram is shown I FIG. 12, while hierarchical high speed (HS) data structure is shown in FIG. 13. In summary, I-WIN clinical work includes real-time high-speed big data acquisition, real-time waveform and summarized data view and early and accurate prediction of clinical events, while research work includes high-speed high-performance data warehouse and real-time AI development platform.

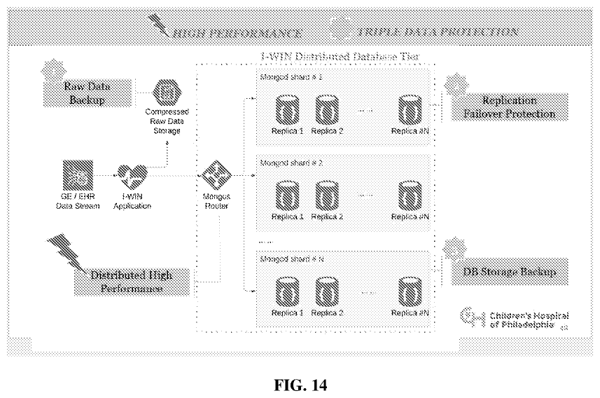

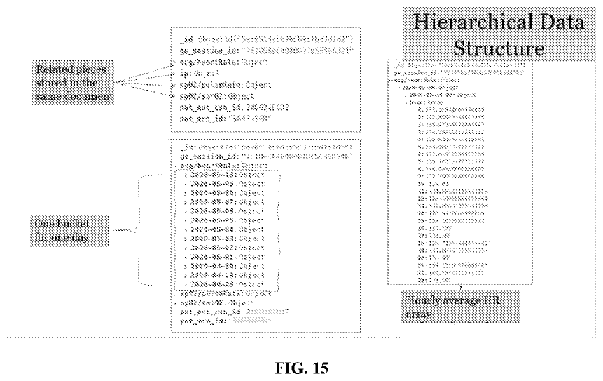

As shown in FIG. 14, I-WIN provides data protection in the forms of raw data backup, replication failover protection and database storage backup. From a clinical care perspective, I-WIN can reduce care provider burdens, e.g. through tele-health for enabling remote patient monitoring with bundled data view for clinicians, early warning of patients at risk (4-hour prior to an event with AUC>94%), and automated data recordings of vital signs. In addition, I-WIN can improve quality of patient care through critical event reduction and morbidity and mortality rate reduction. From a research perspective, I-WIN provides a data query platform for high-speed vital signs (DQP-H) retrieval, including waveforms and provides a real-time Data & AI platform (DAP-R) for customized models. FIG. 15 illustrates an example of coding for I-WIN hierarchical data structure, showing related pieces are stored in the same document and an hourly average heart rate array.

As disclosed herein, I-WIN provides real-time data collection from (hospital-wide) bedside monitors, event-driven analysis, AI-based probabilistic models for risk prediction, personalized risk explanation and personalized intervention strategies. In addition, I-WIN provides integration of structured, unstructured, high-frequency patient data, as well as web-based user interface with unique integrated data view.

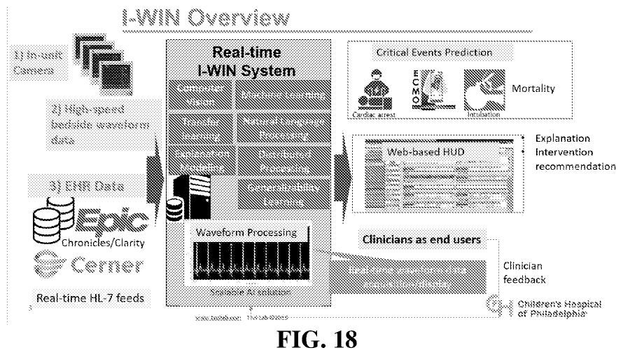

Referring now to FIG. 18, an overview of an exemplary embodiment of the I-WIN system is provided. In the embodiment, the system includes an in-unit camera and utilizes high-speed bedside waveform data and EHR data. Additional details are shown and described in the figure.

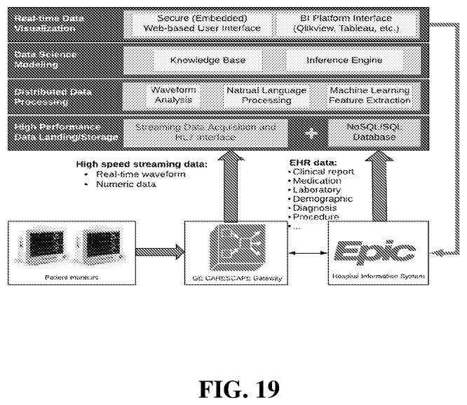

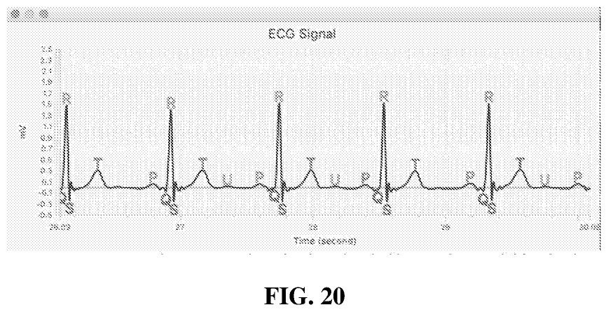

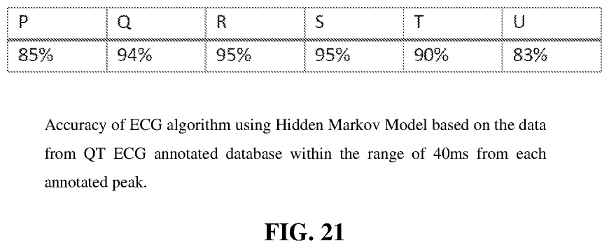

Referring now to FIG. 19, the proposed architecture of an exemplary embodiment of the present disclosure is shown. This embodiment includes real-time data acquisition and storage, signal processing, natural language processing, predictive modeling with explanation and transfer learning, and user interface. The embodiment of FIG. 18 illustrates the Intensive-care Warning Index (I-WIN) architecture comprises multiple components, including real-time high-performance data collection, storage and data integration, data processing, data analytics, and data visualization. The data acquisition module for collecting and storing HSD can be developed and evaluated via a scalable and robust real-time tool. The proposed pilot hardware and software will be scalable for future demand (e.g., hospital-wide data collection and storage) without replacing the current setup but adding additional distributed nodes (servers). Specifics of proposed work are provided in the attached Examples.

Exemplary embodiments may include in-house development in conjunction with various inhouse/vendor support, and the deployment environment can be a Research IS Network. In specific embodiments, the hardware may include two or more servers [e.g. Dell R740 each with GPU (1), 24-core CPU (1), 512 GB memory (1)] and tiered EMC Isilon storage (e.g. initial 200 TB with tiered storage and backup).

In particular embodiments, the operating system may include Red Hat Enterprise Linux (RHEL), and the AI platform may include TensorFlow, Java and Python. The software may utilize knowledge base and inference engines. Cluster management software may include Apache Kafka, Apache Spark data streaming platform, Database: MongoDB [open source]), Postgres [open source] and/or Python [open source].

### Example 1—Provide Data Acquisition Module for Collecting and Storing HSD

The inventors collect two types of high-speed data from a GE network: waveform (with sampling rates up to 240 Hz) and numeric data (with sampling rates up to 1 Hz). The waveform data from a bedside monitor comprises a total of 9 channels including 7 channels of electrocardiogram (ECG) data with a sampling rate of 240 Hz per channel, one channel of blood pressure (BP) with a sampling rate of 120 Hz and one channel of peripheral capillary oxygen saturation (SpO2) with a sampling rate of 60 Hz. The numeric data comprises more than 100 variables such as respiratory rate, heart rate, etc. sampled at the second level.

To provide a scalable solution for data collection and data storage, the inventors have developed a distributed platform following industrial (open source) standards for both hardware and software design. With over more than one year of system deployment at CHOP, the inventors have evaluated the data storage and are confident to have enterprise-level of services, quality, and sustainable maintenance.

The inventors have collected a total of 316 beds from ICUs, their stepdown care unit (CCU), and floor units. The inventors' design is scalable for hospital-wide monitors (around 600 beds).

The inventors conducted HSD evaluation: comparing the inventors' collected data with the BedmasterEx™ database, a FDA approved system. The inventors computed the mean square errors (MSE) between the inventors' data stream and an existing data stream (from BedmasterEx™). The results show both the BedmasterEx™ and the inventors' system received exactly the same HSD and the MSE were zero.

### Example 2—Provide Signal Processing Models for Extracting Features from HSD

The inventors previously developed wavelet application for vital sign time-series prediction.[14] The inventors have recently developed advanced ECG processing algorithms (including wavelet transforms, hidden Markov models, digital filters) that can identify six ECG waves (P, Q, R, S, T and U waves) and ST segment (elevation or depression). FIG. 19 shows the screenshot of the inventors' developed algorithm in Tsui Lab. Table 1 shows the inventors' pilot evaluation results when applying the inventors' algorithm to the publicly available QT database[15] with annotated ECG data.

To extract features from waveform data, the inventors have developed signal processing tools to the proposed architecture, which can process all ECG channels from collected beds. The waveform features extracted from the received waveform data can be further fed into the inventors' predictive models and database in the inventors' architecture.

For blood pressure waveforms, the inventors will extract features such as systolic and diastolic blood pressures, mean blood pressure, etc. For SpO2 waveforms, the inventors will extract waveform characteristics such as mean, maximum, and minimum amplitude values. Similarly, those extracted data will be fed into the predictive models and database in the inventors' architecture.

The inventors evaluated the outcomes of the signal processing methods. The inventors conducted two evaluation approaches: 1) compared the inventors' collected waveform data with GE numeric data through averaging the waveform data to the second level, computing ST segments, heart rates, etc.; 2) compared the inventors' identified ECG waves (e.g., P, Q, R, etc.) with cardiologists' annotated ECG waveform results. The evaluation metrics include the mean square errors, sensitivity, specificity, and accuracy. The inventors' system demonstrated high performance outcomes.

### Example 3—Provide Predictive Models with Explanation for Critical Events from EHRs and HSD

The inventors have developed predictive models using Bayesian networks for the prediction of critical events (ECMO, EEI and CA) among patients with single ventricle physiology (93 patients with 131 critical events). The pilot prediction performance reached up to AUC 0.86 (95% C.I.: 0.84-0.88) and the inventors' study manuscript has been accepted by the Journal of Thoracic and Cardiovascular Surgery (JTCVS).[1] The inventors have recently further improved the inventors' methods by developing advanced algorithms (recurrent deep neural networks (Long-short term memory), frequent temporal pattern analysis, and random forest) and the latest performance up to AUC 0.91 (95% C.I.: 0.88-0.95). The developmental work was based on the data collected from the UPMC Children's Hospital of Pittsburgh (CHP).

For EHR data collection, the inventors had experience working on retrieving patient records from commercial EHR systems such as Epic and Cerner. The inventors have developed different communication channels (e.g., web series and HL7) to retrieve EHR system data and various hospital information systems. The data types the inventors include but not limited to demographics, encounter visit registration data, laboratory tests, medications, and clinical narrative reports. To process clinical narrative reports (unstructured data) such as history and physical exam (H&P) and progress notes (PN), the inventors have developed NLP tools to extract clinical concepts including findings and symptoms from those reports. One of the inventors' developed NLP algorithm won top 4 NLP competition in 2016 international contest with 23 teams from academics and industry startups in several counties [16].

To provide explanation for predictive models, the inventors have implemented two state-of-the-art explanation algorithms Local Interpretable Model-agnostic Explanations (LIME)[17,18] and the Shapley Additive explanation (SHAP)[19,20]. The evaluation metrics included risk explanation, prediction accuracy, and runtime. The inventors evaluated the two explanation models in two datasets: 30-day pediatric readmission prediction (our previous work) and infant mortality prediction (500+ cases and 60,000+ controls)[21]. The inventors compared the two explanation models with their previously built predictive models (a naïve Bayes and a ridge regression). The inventors found SHAP algorithm is efficient and providing reasonable and patient-specific explanation based on key features identified by SHAP.

This aim is the main core of the proposed project. The inventors will collect historical EHR data (mainly unstructured data) from Epic and clinical data warehouse to build predictive models following the modeling framework the inventors previously developed. The inventors will apply the inventors' developed NLP tools to extract features from unstructured data. Since there will be no historical HSD available for the model, the inventors will use expert knowledge (such as ST elevation/depression for the impact of critical events) and embed the knowledge to the models the inventors will develop.

The inventors will work with clinicians to develop SHAP explanation algorithm for prediction explanation. The inventors plan to group actionable variables (risk factors) in one category and non-actionable variables in another group.

To evaluate the outcomes of the work in Aim 3, the inventors will first evaluate model performance based on 10-fold cross validation. The evaluation metrics include AUC, sensitivity, specificity, positive predictive value (PPV), and timeliness. The gold standard is the events identified in the Epic system. For unit test of the predictive outcomes computed from the AI platform, the inventors will compare them with the answers the inventors obtained from the inventors' development environment to ensure the outcomes between the proposed distributed platform and the development platform in Tsui lab are the same. For explanation evaluation, the inventors will work with clinicians to evaluate the quality of the explanation based on a 5-point liker scale survey.

### Example 4—Provide Data Visualization Module for Viewing HSD, EHR and Outcomes

The inventors previously developed a graphical user interface (GUI) embedded in Cerner EHR system to display 30-day hospital readmission risk for each inpatient. The inventors also previously conducted stake holder meetings to ascertain the need of end users (clinicians and nurses) for other IT deployment projects [22].

The inventors have first created stakeholder group (including clinicians and nurses) and conducted stakeholder meetings to identify key information needed to be displayed on the GUI for visualization. The development team form several mock-up pages to demonstrate the proposed ideas to the stakeholder group. Several iterations between the development team and the stakeholder group have made to finalize the GUI.

The inventors have worked with stakeholder group to evaluate the quality of the display based on a 5-point liker scale survey.

### Example 5—Deploy and Evaluate I-WIN System

The inventors previously had experience in deploying a production system at CHP through a standard 3-stage management process, i.e., alpha (or laboratory) environment, beta (or semi-production) environment, and production environment [23]. The alpha environment allows developers to develop and test the system. The beta environment has the same data seen in the production environment but no real users like clinicians or nurses will be affected. The 3-stage management process is designed to minimize unexpected impact to the production environment. The inventors also developed system maintenance manuals for IT team to perform routine maintenance and worked with the IT team to resolve any issues identified.

The inventors first test the entire system in an alpha environment with an end-to-end approach by feeding stream waveform data and EHR data to the predictive models and show the output of the system through the proposed GUI. The inventors use dockers to manage each of the 3 stages. After the internal test is completed in the alpha environment, the inventors then promote it to the next stage. The inventors work with hospital IS teams to follow their standard deployment process.

To evaluate the deployment of the system, the inventors first identify any issues from the pilot users on the beta environment. The inventors conduct the beta evacuation for about one month before the inventors deploy it to production. The inventors then compare the results between the beta and production environments.

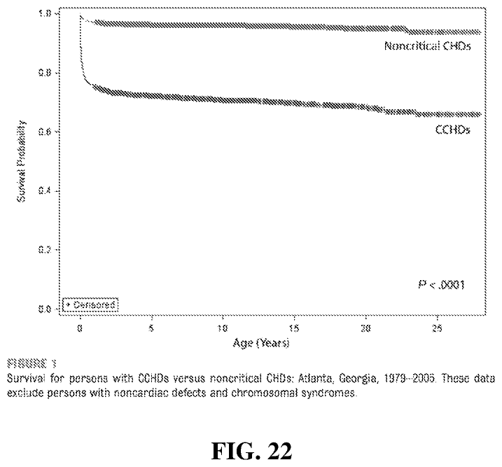

FIG. 22 is extracted from Oster [29] illustrates the survival probability for persons with CCHD versus noncritical CHDs over different ages.

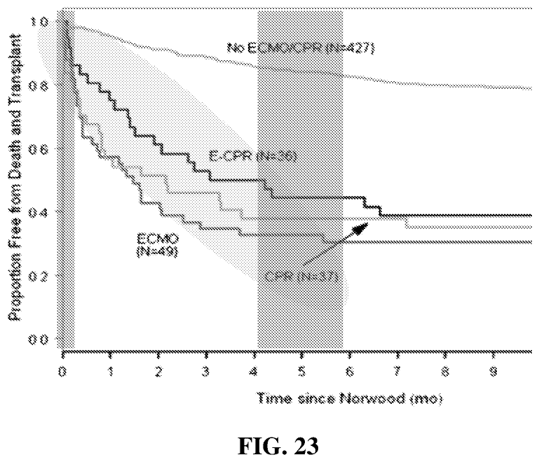

FIG. 23 is adapted from Tebbutt [2] and illustrates the proportion free from death and transplant versus time as noted.

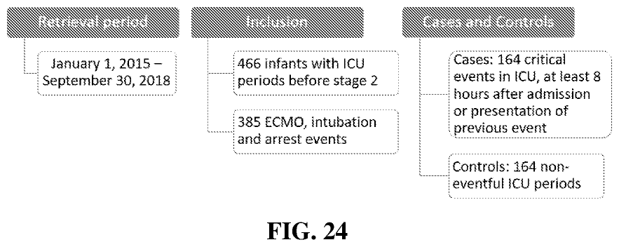

Referring now to FIG. 24, data retrieved from the Children's Hospital of Philadelphia

(CHOP) included routinely-collected physiological variables and laboratory-test results identified by intensivists as relevant for the prediction of patient deterioration, e.g., heart rate, blood pressure, oxygen saturation, creatinine, lactate, base excess.

Specific exemplary embodiments of the present disclosure are configured to achieve early and accurate prediction of deterioration in single-ventricle infants in the perioperative period before stage-2 palliation. It is believed that leveraging longitudinal changes in routinely-collected, physiological data may help predict the onset of critical events with up to eight hours of anticipation.

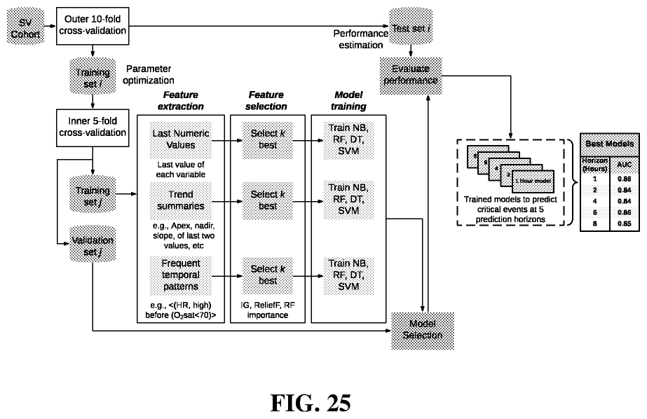

FIG. 25 illustrates a flowchart of one embodiment including parameter optimization, feature extraction, feature selection, and model training.

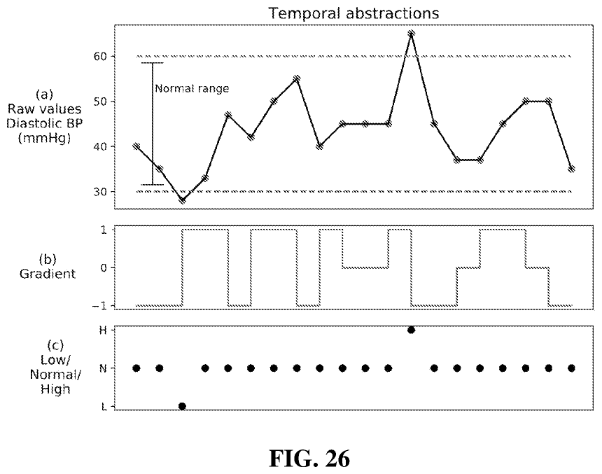

FIGS. 26-28 are published from Shahar [30] and illustrate Frequent Temporal Patterns (FTPs).

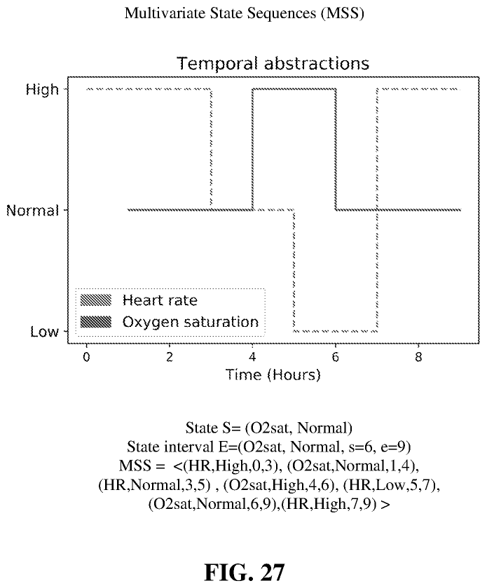

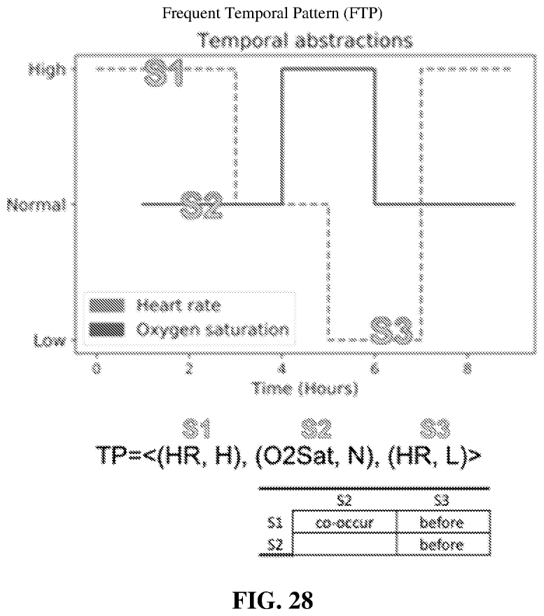

FIG. 26 illustrates temporal abstractions, e.g. with gradient (change), slope, time since measurement, low/normal/high. FIG. 27 shows multivariate state sequences (MSS) as noted. In FIG. 28, the Frequent Temporal Pattern (FTP) is shown for heart rate and oxygen saturation. The state intervals' start/end times are abstracted into temporal relationships between states (before, co-occur, after).

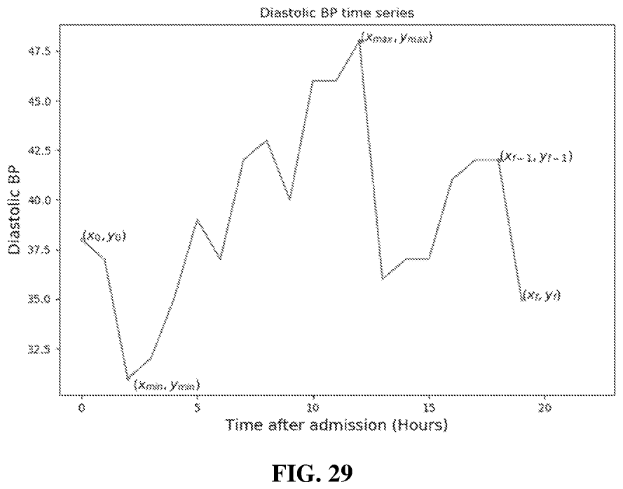

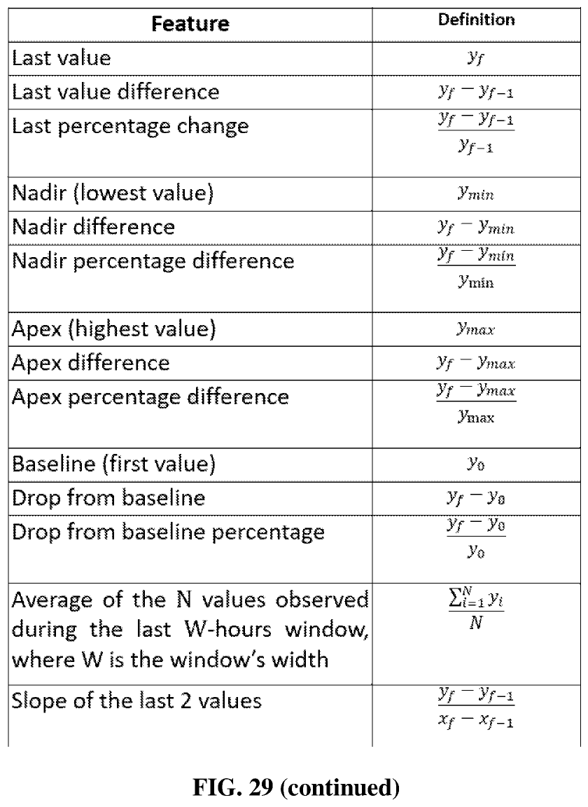

FIG. 29 is published form Valka [31] and illustrate trend (temporal features) as noted.

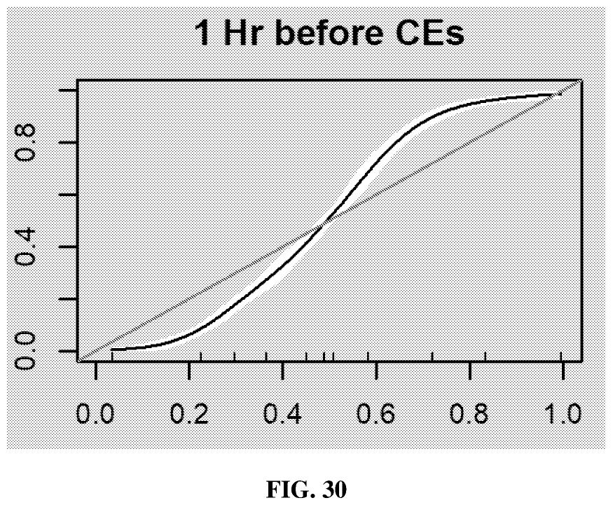

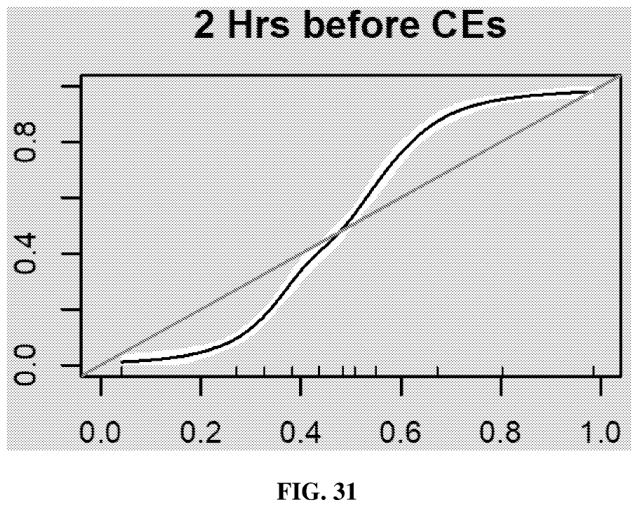

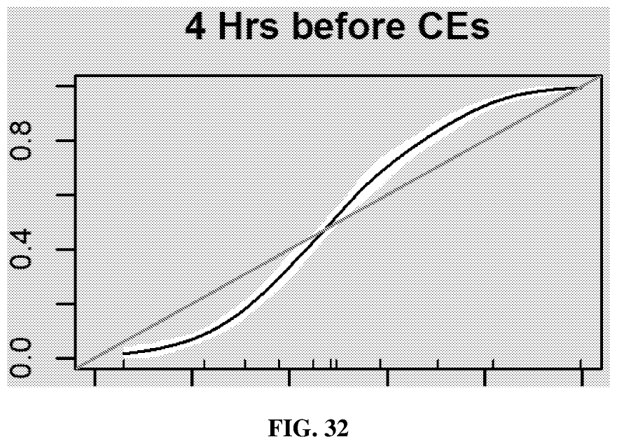

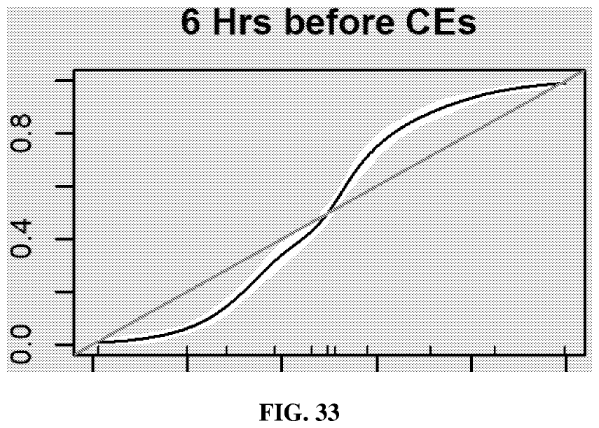

FIGS. 30-33 display results of exemplary embodiments, including calibration curves of FTP models at different times before critical events.

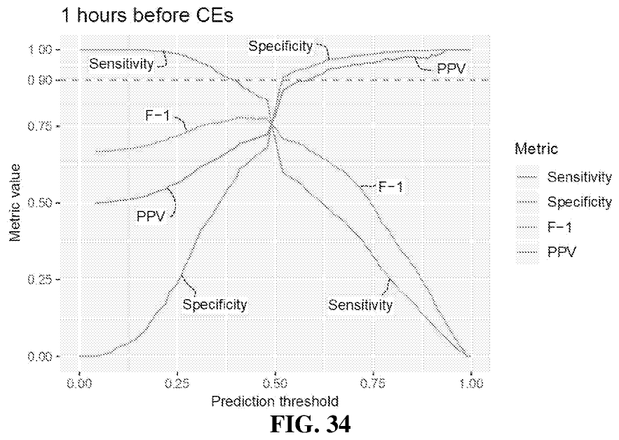

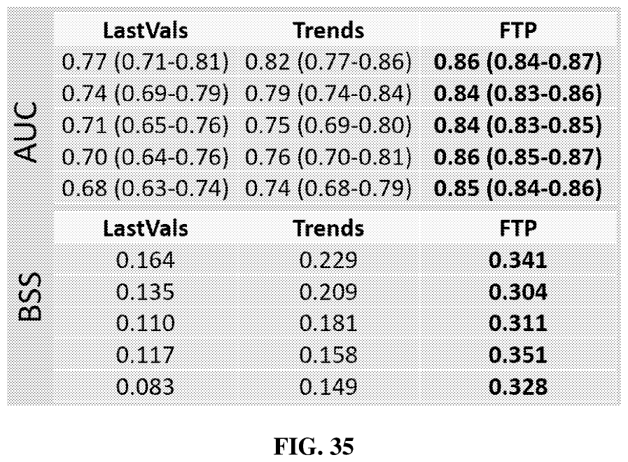

FIG. 34 displays characteristics of a best FTP model, and FIG. 35 displays last values, trends and FTP as noted.

The inventors leveraged the temporal information in longitudinal, objective data from SV infants admitted to the ICU to meet the need of accurate and early prediction of critical events. Predictors can be extracted automatically from EHR systems and are routinely collected. Early prediction of critical events may enable clinicians to target interventions aimed at reducing morbidity, mortality, and healthcare costs associated with SV defects.

Accordingly, exemplary embodiments of the present disclosure provide significant benefits and advantages to both patients and care providers.

All of the devices, systems and/or methods disclosed and claimed herein can be made and executed without undue experimentation in light of the present disclosure. While the devices, systems and methods of this invention have been described in terms of particular embodiments, it will be apparent to those of skill in the art that variations may be applied to the devices, systems and/or methods in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

The contents of the following references are incorporated by reference herein:
1. Ruiz V, Saenz L, Lopez-Magallon A, Shields A, Ogoe H A, Suresh S, Munoz R, and Tsui F. Early prediction of critical events for infants with single ventricle physiology in critical care using routinely collected data. J Thorac Cardiovasc Surg. 2019; 158(1):234-243.e3. doi:10.1016/j.jtcvs.2019.01.130.
2. Tabbutt S, Ghanayem N, Ravishankar C, Sleeper L A, Cooper D S, Frank D U, Lu M, Pizarro C, Frommelt P, et al. Risk factors for hospital morbidity and mortality after the Norwood procedure: A report from the Pediatric Heart Network Single Ventricle Reconstruction trial. J Thorac Cardiovasc Surg. 2012. doi:10.1016/j.jtcvs.2012.05.019
3. Chen J, Bellomo R, Flabouris A, Hillman K, Assareh H, and Ou L. Delayed emergency team calls and associated hospital mortality: A multicenter study. Crit Care Med. 2015; 43(10):2059-2065. doi:10.1097/CCM.0000000000001192
4. Opio M O, Nansubuga G, and Kellett J. Validation of the VitalPAC™ Early Warning Score (ViEWS) in acutely ill medical patients attending a resource-poor hospital in sub-Saharan Africa. Resuscitation. 2013; 84(6):743-746. doi:10.1016/j.resuscitation.2013.02.007
5. da Silva Y S, Hamilton M F, Horvat C, Fink E L, Palmer F, Nowalk A J, Winger D G, and Clark R S B. Evaluation of Electronic Medical Record Vital Sign Data Versus a Commercially Available Acuity Score in Predicting Need for Critical Intervention at a Tertiary Children's Hospital. Pediatr Crit Care Med. 2015; 16(7):644-651. doi:10.1097/PCC.0000000000000444
6. Downey C L, Tahir W, Randell R, Brown J M, and Jayne D G. Strengths and limitations of early warning scores: A systematic review and narrative synthesis. Int J Nurs Stud. 2017; 76:106-119. doi:10.1016/j.ijnurstu.2017.09.003
7. Rusin C G, Acosta S I, Shekerdemian L S, Vu E L, Bavare A C, Myers R B, Patterson L W, Brady K M, and Penny D J. Prediction of imminent, severe deterioration of children with parallel circulations using real-time processing of physiologic data. J Thorac Cardiovasc Surg. 2016; 152(1):171-177. doi:10.1016/j.jtcvs.2016.03.083
8. Kappen T H, Van Loon K, Kappen M A M, Van Wolfswinkel L, Vergouwe Y, Van Klei W A, Moons K G M, and Kalkman C J. Barriers and facilitators perceived by physicians when using prediction models in practice. J Clin Epidemiol. 2016; 70:136-145. doi:10.1016/j.jclinepi.2015.09.008
9. Simpao A F, Ma A A, Tan J M, Wasey J O, Masino A J, and Galvez J A. One Laryngospasm, 2 Realities. A A Pract. 2018; 11(11):315-317. doi:10.1213/XAA.0000000000000817
10. Rajpurkar P, Hannun A Y, Haghpanahi M, Bourn C, and Ng A Y. Cardiologist-Level Arrhythmia Detection with Convolutional Neural Networks. arXiv. 2017. doi:1707.01836
11. Ghassemi M, Celi L A, and Stone D J. State of the art review: The data revolution in critical care. Crit Care. 2015; 19(1). doi:10.1186/s13054-015-0801-4
12. Matam B R, and Duncan H. Technical challenges related to implementation of a formula one real time data acquisition and analysis system in a paediatric intensive care unit. J Clin Monit Comput. 2018; 32(3):559-569. doi:10.1007/s10877-017-0047-6
13. Tsui F-C, Li C-C, Sun M, and Sclabassi R J. Acquiring, modeling, and predicting intracranial pressure in the intensive care unit. Biomed Eng—Appl Basis Commun. 1996; 8(6).
14. Tsui F-C, Li C-C, Sun M, and Sclabassi R J. Adaptive neural network in wavelet space for time-series prediction. In: Proceedings—IEEE International Symposium on Circuits and Systems. Vol 3.; 1996.
15. Physionet.org. The QT database.
16. Posada J D, Barda A J, Shi L, Xue D, Ruiz V, Kuan P-H, Ryan N D, and Tsui F R. Predictive modeling for classification of positive valence system symptom severity from initial psychiatric evaluation records. J Biomed Inform. 2017; 75. doi:10.1016/j.jbi.2017.05.019
17. Katuwal G J, and Chen R. Machine Learning Model Interpretability for Precision Medicine. Arxiv ID 161009045. 2016.
18. MILLER G A, Krause J, Perer A, Ng K, Baehrens D, Schroeter T, Harmeling S, Hansen KHANSEN K, Klaus-Robert uller KLAUS-ROBERTMUELLER C, et al. Model-Agnostic Interpretability of Machine Learning. 2016 ICML Work Hum Interpret Mach Learn (WHI 2016). 2016. doi:10.1145/2858036.2858529
19. Lundberg S, and Lee S-I. A Unified Approach to Interpreting Model Predictions. 2017; (Section 2):1-10.
20. Erion G, Chen H, Lundberg S M, and Lee S-I. Anesthesiologist-level forecasting of hypoxemia with only SpO2 data using deep learning. 2017; (Nips).
21. Yang L, Posada J, Su H-D, Shi L, Mi F, and Tsui F. Using logistic regression to verify completeness of electronic health records for infant mortality analysis. In: AMIA Annual Symposium Proceeding.; 2017.
22. Barda A, Shi L, Urbach A, Suresh S, and Tsui F. Usability and Acceptability of a System to Identify Pediatric Patients at Risk of 30-day Hospital Readmission Prior to Discharge. In: AMIA 2017.; 2017.
23. Tsui F R, Ruiz V, Barda A, Ye Y, Suresh S, and Urbach A. Retrospective and Prospective Evaluations of the System for Hospital Adaptive Readmission Prediction and Management (SHARP) for All-Cause 30-Day Pediatric Readmission Prediction Children's Hospital of Pittsburgh of UPMC, Pittsburgh, Pa. In: AMIA 2017.; 2017. doi:10.1197/jamia.M1552.6.
24. Mahle W T, Forbess J M, Kirshbom P M, Cuadrado A R, Simsic J M, and Kanter K R. Cost-utility analysis of salvage cardiac extracorporeal membrane oxygenation in children. J Thorac Cardiovasc Surg. 2005; 129(5):1084-1090. doi:10.1016/j.jtcvs.2004.08.012
25. Hayman W R, Leuthner S R, Laventhal N T, Brousseau D C, and Lagatta J M. Cost comparison of mechanically ventilated patients across the age span. J Perinatol. 2015; 35:1020-1026.
26. Duncan H P, and Frew E. Short-term health system costs of paediatric in-hospital acute life-threatening events including cardiac arrest. Resuscitation. 2009; 80(5):529-534. doi:10.1016/j.resuscitation.2009.02.018
27. CHOP. Cardiothoracic anesthesiology. https://www.chop.edu/centers-programs/cardiaccenter/cardiothoracic-anesthesiology. Accessed Jan. 23, 2019.
28. CHOP. Cardiology. https://www.chop.edu/services/cardiology. Accessed Jan. 23, 2019.
29. Oster M E, Lee K A, Honein M A, Riehle-Colarusso T, Shin M, Correa A. Temporal Trends in Survival Among Infants With Critical Congenital Heart Defects. Pediatrics. 2013; 131(5):e1502-e1508. doi:10.1542/peds.2012-3435.
30. Shahar Y (1997) A framework for knowledge-based temporal abstraction. Artif Intell.
31. Valko M, Hauskrecht M. Feature importance analysis for patient management decisions. Stud Health Technol Inform. 2010; 160(Pt 2):861-865.\
32. Halpern N A. Critical care statistics. https://www.sccm.org/Communications/Critical-Care-Statistics. Accessed Mar. 24, 2020.

1. A method for early detection and management of clinical critical events, the method comprising:
analyzing high-speed bedside waveform data;
analyzing electronic health record (EHR) data; and
predicting critical events (CE) based on the analysis of the high-speed bedside waveform data and the analysis of the EHR data.

2. The method of claim 1 wherein analyzing high-speed bedside waveform data and electronic health record (EHR) data comprises one or more of the following: computer vision, transfer learning, explanation modeling machine learning, natural process learning, distributed processing, generalizability learning or waveform processing.

3. The method of claim 2 wherein the waveform processing comprises real-time waveform data acquisition.

4. A system for early detection and management of clinical critical events, the system comprising:
a module configured for high performance data landing or storage;
a module configured for distributed data processing;
a module configured for data science monitoring; and
a module configured for real-time data visualization.

5. The system of claim 4 wherein the module configured for high performance data landing or storage comprises a scale-out storage solution and a NoSQL/SQL database.

6. The system of claim 4 wherein the module configured for distributed data processing is configured to perform waveform analysis, natural language processing or machine learning feature extraction.

7. The system of claim 4 wherein the module configured for data science monitoring comprises a knowledge base and an inference engine.

8. The system of claim 4 wherein the module configured for real-time data visualization comprises a secure web-based user interface and a business intelligence (BI) platform interface.

9. A system for early detection and management of clinical critical events, the system comprising:
a data source layer;
an extract, transform and load (ETL) layer;
a distributed artificial intelligence (AI) layer; and
a presentation layer, wherein:
the data source layer is configured to transfer data to the ETL layer;
the ETL layer is configured to transfer data to the distributed AI layer; and
the distributed AI layer is configured to transfer data to the presentation layer.

10. The system of claim 9 wherein the data source layer comprises medical devices and bedside monitors.

11. The system of claim 10 wherein the ETL layer includes streaming data acquisition from the bedside monitors and the medical devices.

12. The system of claim 11 wherein the data source layer comprises:
an electronic health record (EHR) system;
an administrative information system; and
a clinical information system.

13. The system of claim 12 wherein the ETL layer is configured to apply natural language processing to data transferred from the EHR system, the administrative information system and the clinical information system.

14. The system of claim 13 wherein the ETL layer is configured to clean data from the data source layer via natural language processing.

15. The system of claim 13 wherein the ETL layer is configured to merge records from the data source layer via natural language processing.

16. The system of claim 13 wherein the ETL layer is configured to apply business rules to the data source layer via natural language processing.

17. The system of claim 13 wherein the distributed AI layer comprises a hybrid database cluster.

18. The system of claim 17 wherein the distributed AI layer comprises predictive models and an inference engine configured to communicate with the hybrid database cluster.

19. The system of claim 18 wherein the distributed AI layer is configured to apply signal processing the streaming data acquisition from the ETL layer.

20. The system of claim 19 wherein the presentation layer comprises a web application and graphical user interface.
